(19) United States
(12) Patent Application Publication
(10) Pub. No.: US 2026/0334847 A1
(43) Pub. Date: Oct. 29, 2026

(54) Detection system of electroencephalographic signals

(71) Applicant: Spes Medica Srl, Genova (IT)

(72) Inventors: Matteo Mistretta, Genova (GE) (IT); Elena Cotella, Bogliasco (GE) (IT); Patrick Phelan, Newburyport, MA (US)

(21) Appl. No.: 19/712,559

(22) Filed: Jun. 18, 2026

Related U.S. Application Data
(63) Continuation-in-part of application No. 16/380,290, filed on Apr. 10, 2019, now abandoned.

(30) Foreign Application Priority Data
Apr. 12, 2018 (IT) 102018000004455

(51) Int. Cl. A61B 5/369; A61B 5/257

(52) U.S. Cl. CPC A61B 5/369; A61B 5/257; A61B 2562/14

(57) ABSTRACT

A system for detecting electroencephalographic signals includes at least one electrode for the transmission of the detected signal, a conduction system of the signal, which is interposed between the electrode and the scalp of a patient, and a fixation system of the electrode to the scalp. The conduction system and the fixation system may be provided as a single layer of hydrogel.

### FIELD OF THE INVENTION

The present invention relates to a system for detecting electroencephalographic signals comprising at least one electrode for transmission of the detected signal, a conduction system of the signal interposed between the electrode and the scalp of a patient, and a fixation system of the electrode to the scalp.

### BACKGROUND OF THE INVENTION

In the typical configuration of state of the art systems that are commonly used to detect and record electroencephalographic signals (EEG), the recording of EEG signals is a non-invasive practice, involves the positioning of one or more electrodes in contact with the scalp of a patient through an electrolyte, for transmitting the signal to a processing unit.

The signal is detected by the electrode thanks to the presence of a layer of conductive gel interposed between the electrode and the scalp.

In view of the nature of the detected signal, it is evident that a correct registration and subsequent processing of EEG signals require a firm positioning of the electrode so as to provide the processing unit with accurate data to avoid the generation of artifacts, or inaccurate readings that generate an incorrect analysis.

To ensure a correct positioning of the electrode, the systems known in the art use adhesives, creams, etc., which provide a firm fixation in the desired position.

One of the major disadvantages in the use of these adhesives, creams, etc. is related to the cleaning of the scalp of the patient after their use: in fact, the removal of adhesivess, creams, etc. is not particularly effortless, easy and atraumatic and requires a careful cleaning.

In systems known in the state of the art, the cleaning procedure on the scalp is necessarily carried out with detergent materials and/or solvents, not always suited to the scalp.

In fact, the used solvents must be particularly aggressive in order to obtain a satisfactory cleaning the scalp which allows the removal of the used adhesives and creams.

These effects are particularly troublesome for the patient if, as happens in common practice, the recording of EEG signals requires the use of numerous electrodes, arranged along the entire surface of the skull of the patient.

Therefore, in systems known in the state of the art, the procedure for cleaning and removing the electrodes has no negative aspects which are just related to the harshness of the used solvents, but also disadvantages in terms of timing.

These disadvantages are found not only in the cleaning process but also in the part preceding the examination and preparation of the patient, i.e. the positioning of the electrodes.

In fact, the use of conductive gel in combination with adhesives for the positioning of the electrodes requires a long duration positioning procedure.

Some systems known in the state of the art seek to avoid the use of adhesives through the positioning of the electrodes supported by elastic material.

However, these electrodes require, in addition to the use of elastic caps that cover the skull of the patient, in order to fix the electrode in position, also the use of liquid gel for the contact with the scalp and the creation of the registration interface.

It is evident that this solution, in addition to increasing the structural complexity of the whole system, requires the availability of owning a range of different sizes of caps that can cover as much as possible the different patients and set-up requires a particularly long configuration time of the entire system.

Therefore, there is a need, not satisfied by the systems known in the art, to provide a detection system of EEG signals which overcomes the disadvantages described above, ensuring a simple and functional positioning of the electrodes.

### SUMMARY OF THE INVENTION

The present invention achieves the above objects by providing a system as described above, in which the conduction means and the fixation means are constituted by a single hydrogel layer.

A particularly dense, nearly solid polymer is made, and has both conductive and adhesive features, so as to speed up the electrode positioning process, using a single component.

The hydrogel may for example be provided in the form of a drop/tablet contained in a dispenser/blister pack capable of allowing a user to use and position the hydrogel on the scalp of the patient in points of interest through the use of, for example, a cup electrode.

According to a preferred embodiment, the hydrogel material is provided in the form of a drop.

According to this configuration, the hydrogel is therefore pre-dosed, i.e. provided in predetermined doses, sufficient to conduct the signal and to ensure a sufficiently wide surface for gluing.

The particularly dense nature of the hydrogel allows to create finite elements that can be taken one by one to be positioned and removed easily from the scalp of the patient.

It is specified that some embodiments illustrated in the following will describe a specific embodiment of the shape of the drop.

The removal of these drop leaves minimal residue on the hair of the patient, easily removable with aqueous solutions, not harmful to the scalp of the patient.

The shape of the drop also has a specific advantage, i.e. to provide a sort of bearing interposed between the scalp and the electrode, preventing the trauma due to the pressure of the electrode on the scalp, effect known with the name of “skin-breakdown”.

For this reason, the hydrogel that constitutes the drop must have a density such as to be sufficiently rigid for the creation of finite elements, but at the same time sufficiently soft, i.e. mechanically yielding, to change its surfaces so as to adapt them to the contact surfaces of the skull of the patient and/or the electrode, once the drop is used for the detection of the signals.

Moreover, the hydrogel that constitutes the drop also has both conductive and adhesive features with respect to the skull of the patient.

A system according to the present invention can involve any type of electrode known in the state of the art, which has a surface facing the scalp adapted to house said drop.

The shape of the drop is particularly advantageous in combination with the cup electrodes, especially if the drop has a size such as to be housed inside the electrode cup.

This configuration has advantageous effects also on the positioning of the electrodes: the cup electrode can be used by a user in a similar manner to a spoon, which collects the drop to then be immediately placed on the scalp of the patient.

The above-mentioned advantages are particularly relevant in the case in which a plurality of electrodes and a plurality of drops are provided.

Moreover, in order to facilitate the collection of such drops by the cup electrodes, it is possible to group all the drop through a single support element, such as a plate-like support element, on which the drops are placed.

The hydrogel elements remain attached to the upper surface of the plate-like element and can be removed from the electrode cup, to ensure their positioning on the scalp of the patient.

Since the electrode has a wire for the connection of the processing unit, in order to prevent accidental tractions of the clinician with the wire from moving the electrode, an embodiment variant of the system according to the present invention provides retention means for retaining the electrode in position.

For example, such retention means can be constituted by an adhesive support, placed in contact with the scalp of the patient and the outer surface of the head of the electrode.

In addition, in the light of the features described above, the present invention relates to a disposable electrode for detecting electroencephalographic signals that comprises a cup electrode and a hydrogel drop inserted inside the cup of said electrode.

It is therefore possible to provide a single pack of a disposable electrode already ready to be positioned on the scalp of a patient.

As for the embodiment variants described above, also in this case it is possible to provide an adhesive support for retaining the electrode in position on the scalp of the patient.

In the light of the advantageous features described heretofore, the present invention also relates to a method for the detection of electroencephalographic signals.

This method preferably provides for the use of the system or of the electrode described above and in particular comprises the fixation of at least one electrode to the scalp of a patient for the transmission of the detected electrical signals.

According to a method according to the present invention, the fixation of the electrode includes the following steps:
(a) positioning a drop in contact with the electrode,
(b) bonding of the electrode to the scalp of the patient through the drop,
(c) detection of electroencephalographic signals through the drop.

Even in this case, the drop allows the use of a single component that allows at the same time the conduction of EEG signals, as well as the gluing of the electrode to the head of the patient.

The application of the electrode is therefore immediate, fast, efficient and particularly “clean”.

In combination with this embodiment, preferably the step of positioning of the drop in contact with the electrode, provides for the collection of the drop from the plate-like element and the insertion on said electrode, the electrode consisting of a cup electrode.

To improve the retention in position of the electrode, the step of gluing of the electrode provides a step of retaining the position of said electrode to the scalp of the patient through the use of a preformed adhesive support.

In conclusion, an object of the present invention is a method for creating the conduction and fixation means of the system described above.

This method advantageously comprises the following sub-steps:
creating a plurality of hydrogel drops,
arranging the plurality of drops on a support element.

Finally, it is specified that the invention also relates to a collection of hydrogel elements.

This collection includes hydrogel drops created according to one or more of the features described above and which are arranged on a plate-like element, in a similar manner to as some types of tablets or pills are inserted inside pharmaceutical blister packs, as will be illustrated later.

### BRIEF DESCRIPTION OF THE DRAWINGS

These and other features and advantages of the present invention will become clearer from the following description of some exemplary embodiments illustrated in the attached drawings in which:

FIGS. 1a and 1b show two views of a system according to the present invention in one embodiment;

FIG. 2 shows a sectional view of a tablet element coupled to an electrode belonging to a system according to the present invention;

FIG. 3 is a block diagram outlining a method according to the present invention.

### DETAILED DESCRIPTION OF EMBODIMENTS OF THE INVENTION

The figures illustrate a few embodiments of a system and method according to the present invention, in order to better understand its advantages and the described features.

Those embodiments are therefore to be understood as purely illustrative and do not limit the scope of the inventive concept of the present invention, namely, creating a system for detecting electroencephalographic signals by fixing at least one electrode to the scalp of a patient using a single layer of hydrogel and by ensuring both the bonding of the electrode to the scalp and the transmission of electrical signals from the scalp to the electrode.

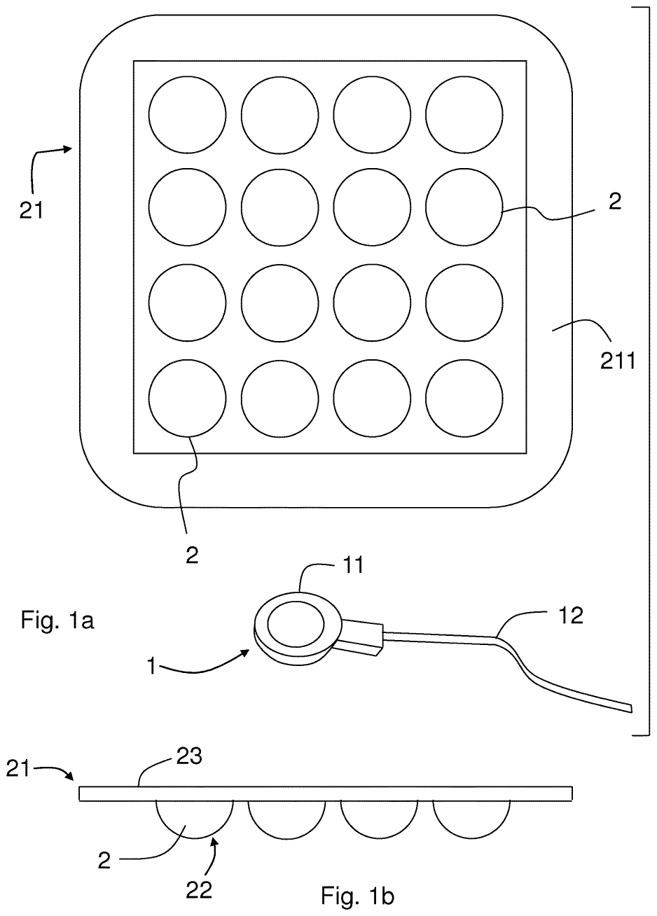

In particular, FIGS. 1a and 1b illustrate a preferred embodiment of a system according to the invention.

In this embodiment, the system comprises at least one electrode 1 for transmitting the detected signal and a conduction system, or means, of the signal interposed between the electrode 1 and the scalp of a patient, as well as a fixation system, or means of the electrode to the scalp.

The fixation means and the conduction means are constituted by a single hydrogel drop 2, which may have different shapes, for example and without limitation, spherical, semi-spherical, cylindrical, or tablet-shaped.

FIG. 1a shows a plurality of drops 2, while showing a single electrode 1.

It is evident that a system according to the invention can comprise any number of electrodes 1 and any number of drops 2.

The electrode 1 may be configured as a cup electrode. with a cup end 11 and a signal transmission cable 12.

The cup end 11 is adapted to be positioned on the skull of the patient, while the transmission cable 12 can transmit the detected signal to a processing unit, provided in an e lectrocardiograph known in the art.

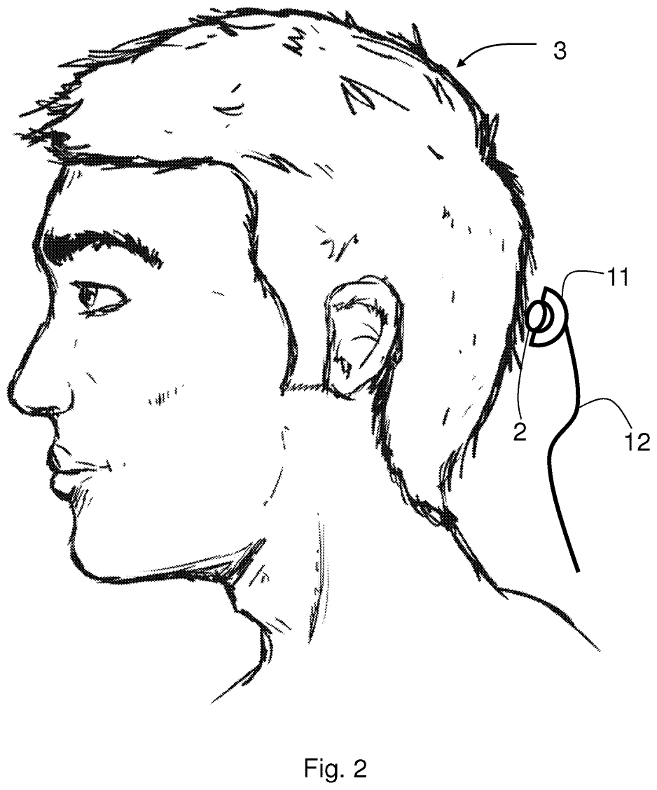

The cup end 11 defined a housing seat for the drop 2, as illustrated in FIG. 2.

FIG. 2 shows a patient 3 and a section of the electrode 1 in combination with the drop 2.

The cup end 11 houses in its interior the drop 2.

For simplicity of illustration, the cross-section of the drop 2 of FIG. 2 is shown as an ellipse, however, being constituted by a hydrogel, the drop 2 can be deformed so as to adhere to the inner surface of the cup end 11 and to the scalp of the patient 3.

This deformation provides for a seamless contact between the scalp of the patient 3 and the cup end 11 of the electrode 1.

Advantageously, the drop 2 has dimensions sufficient for housing inside the cup end 11, without an excessive deformation of the drop 2 that causes an excessive spillage of hydrogel from the edges of the cup ends 11.

Advantageously, the dimensions of the drop 2 are such as to enable it to be housed inside the cup end 11 and cover the edges facing toward the scalp.

In the condition of the positioning of the electrode 1, the drop 2 therefore deforms its surfaces so as to create surfaces complementary to the scalp of the patient 3 and at the cup end 11.

In another embodiment, a retention system, or means, may be provided for retaining the electrode 1 in position, not shown in FIG. 2, and assist the bonding action performed by the hydrogel of the electrode 1 to the scalp of the patient 3.

Such retention system is preferably constituted by an adhesive support, which can be positioned on the outer surface of the cup end 11 and with its ends fixed to the scalp of the patient 3.

According to the variant illustrated in FIGS. 1a and 1b, the system further comprises a plate-like support element 21 on which to place the plurality of drops 2.

The drop 2 are none other than drops of hydrogel arranged on the upper surface of the plate-like element 21, made in the form of a sheet, preferably of plastic material, on which the drops 2 remain fixed thanks to their adhesive feature, until they are removed to be used in combination with the electrodes 1.

It is evident that the plate-like support element can be constituted by a simple sheet of plastic material on which drops 2 can be arranged.

According to the variant illustrated in FIGS. 1a and 1b, the plate-like element is constituted by a blister pack-type element 21 that presents housing seats 22 of the drop 2.

The drop 2 are deposited inside the housing seats 22, after which the blister pack 21 is closed by a protective plastic film 23.

The protective plastic film 23 is attached to the blister pack 21 in such a way that, once removed, the drops 2 remain inside the housing seats 22.

For example, it is possible to provide a zone 211, in which the protective film 23 may be bonded or heat-welded to the blister pack 21.

In alternative, it is possible to envisage that the blister packs 21 is constituted by a flat plate-like element without housing seats, on which the individual drop are arranged.

More generally, a hydrogel drop 2 according to the invention is provided in a dose amount sufficient to conduct the signal and has an outer surface that can adhere to the scalp of a patient.

Further, a hydrogel drop 2 according to the invention has a density sufficient to retain the same predetermined outer shape before and after extraction from the blister pack and to still adapt to the contour of the scalp of the patient.

In one embodiment, hydrogel drops 2 according to the invention have a surface of hemispherical shape, adapted to be housed inside the cup end 11 of the electrode 1.

In one embodiment, the hydrogel drops 2 have a diameter of 12 mm and are provided in blister packs that have a plane dimension of 116 mm×78 mm and that are made from a cardboard backing and a polyethylene terephthalate (PET) cover. In one embodiment, the PET cover has a thickness of 0.6 mm, so that a clinician may be able to push the hydrogel drop from the blister pack without excessive effort.

In a similar manner it is possible to provide a disposable electrode, preferably a cup electrode having a hydrogel drop 2 inserted inside the cup end 11.

The cup end 11 can, for example, be sealed by a protective film in a manner similar to the protective film 23 described previously, in this case fixed to the edges of the cup end 11.

In one embodiment, illustrated in FIGS. 1a and 1b, the blister pack 21 may have a square shape, with sides equal to 140 millimetres.

The housing seats 22, constituted by semi-circular elements, may have a diameter comprised between 20 and 22 millimetres and are spaced by a measure between 3 and 5 millimetres.

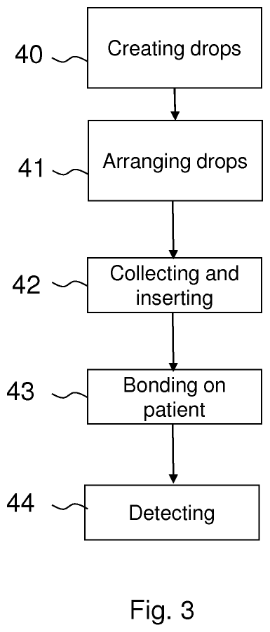

A system according to the invention may be used to perform a method for detecting electroencephalographic signals, of which a possible embodiment is illustrated in FIG. 3 through a flow chart.

As in the methods known in the art, a method according to the present invention involves the fixation of at least one electrode to the scalp of a patient for the transmission of the detected electrical signals.

From a general point of view, a method according to the present invention includes the following steps:
(a) positioning a drop in contact with the electrode,
(b) bonding the electrode to the scalp of the patient through the drop,
(c) detection of electroencephalographic signals through the drop.

The hydrogel drop 2 performs the dual function of bonding the electrode and conducting the signal.

In the embodiment illustrated in FIG. 3, based on the system illustrated in FIGS. 1a, 1b and 2, a method according to the present invention can be used with a plurality of electrodes, in order to position the electrodes on a wide surface of the skull of the patient and to obtain the tracking of the EEG signal.

In particular, the fixation of the electrodes includes the following steps:
collecting and inserting each drop on a corresponding electrode 1, step 42,
bonding the electrode 1 to the scalp of the patient 3, step 43, and
detecting the signal, indicated with 44.

The step indicated with 42 is preferably provided in combination with the use of the cup electrode 1.

Because the drops 2 are made from an adhesive hydrogel, the drops 2 are fixed to the plate-like element 21 in a stable manner.

When a clinician must secure the electrode 1 to the scalp of a patient, due to the peculiar shape of the cup electrode 1, the clinician will not have to handle the drop 2, but it will be sufficient to use the cup end 11 as a spoon to remove the drop 2 from the plate-like element or from the blister pack 21 and to receive it into the cup end 11 so as to make contact with the inner surface of the cup end 11.

After the coupling between the drop 2 and the electrode 1 is achieved, the clinician positions the electrode 1 in the region of interest of the skull of the patient 3 using a slight pressure, which causes the deformation of the drop 2, causing the electrode 1 to become bonded to the scalp of the patient 3.

At this point it is possible to detect the electroencephalographic signals to perform the examination of interest.

As known in the art, at the beginning of the method, a step may be performed that is related to the preparation of the skin of the patient, such as, for example, cleaning the electrode is to be positioned.

Finally, in step 43, an additional fixation of the electrode 1 to the scalp of the patient 3 may be provided using an adhesive support, as previously described.

In particular, FIG. 3 illustrates a possible embodiment, which provides a method of use of a system according to the invention and a method for the creation of a system according to the invention.

The method of use is the one just described, in which an clinician uses the packaged element constituted by the drops, inserted inside the blister packs of FIG. 1b, and places the drops on the electrode and carries out the examination.

This “clinician-side” methodology, defined by steps 42 to 44, may be provided in combination with the method for the creation of the fixation and conduction means belonging to a system according to the present invention.

With reference to FIG. 3, this method includes the following steps:
creating a plurality of hydrogel drops 2, indicated with 40,
arranging the drops 2 on a plate-like support element, or inside the housing seats 22 of the blister pack 21, indicated with 41.

The steps from 40 to 44 may be provided in combination, or it is possible to provide that the steps 40 and 41 are performed by a producer of hydrogel or the like, while steps 42 to 44 can be performed by the users, i.e. by clinicians who intend to perform EEG signal detection analysis.

While the invention can be changed according to different modifications and alternative constructions, some preferred embodiments were shown in the drawings and described in detail.

It should be understood, however, that there is no intention of limiting the invention to the specific illustrated embodiment but, on the contrary, it aims at covering all modifications, alternative constructions, and equivalents falling within the scope of the invention as defined in the claims.

The use of “for example”, “etc.”, “or” refers to non-exclusive non-limiting alternatives, unless otherwise stated.

The use of “includes” means “includes but not limited to”, unless otherwise stated.

The invention claimed is:

1. A system for detecting electroencephalographic signals, the system comprising:
an electrode for transmission of a detected signal;
a conduction and fixation system of the electrode configured to be interposed between the electrode and the scalp of a patient; and
a blister pack containing one or more hydrogel drops,
wherein the conduction and fixation system is constituted of a single hydrogel drop provided in a dose amount sufficient to conduct the signal and having an outer surface sufficient for adhering to the scalp of the patient, the single hydrogel drop having a density sufficient to retain a same predetermined outer shape before and after extraction from the blister pack and to adapt to a contour of the scalp of the patient,
wherein the electrode is disposed within a cup-shaped housing, and
wherein the single hydrogel drop is shaped to be received within the cup-shaped housing.

2. The system according to claim 1, wherein the blister pack has a plurality of blister bubbles each containing one of the hydrogel drops.

3. The system according to claim 1, further comprising retention means adapted to retaining the electrode in position on the scalp of the patient.

4. A method of detecting encephalographic signals, the method comprising:
providing a system for detecting electroencephalographic signals according to claim 1; and
fixing the electrode to the scalp of a patient to transmit detected electrical signals,
wherein the fixing includes the following steps:
(a) positioning the single hydrogel drop within the cup-shaped housing;
(b) bonding the electrode to the scalp of the patient, the single hydrogel drop being diposed between the electrode and the scalp; and
(c) detecting electroencephalographic signals through the single hydrogel drop.

5. The method according to claim 4, wherein step (a) further comprises collecting the single hydrogel drop from the blister pack.

6. The method according to claim 5, wherein the blister pack has a plurality of blister bubbles each containing one hydrogel drop.

7. The method according to claim 4, wherein step (b) includes a step of retaining the electrode in position on the scalp of the patient with an adhesive support.
